(19) United States
(12) Patent Application Publication
(10) Pub. No.: US 2025/0352237 A1
(43) Pub. Date: Nov. 20, 2025

(54) HELICAL DEBULKING TOOL WITH CUTTER

(71) Applicant: KONINKLIJKE PHILIPS N.V., EINDHOVEN (NL)

(72) Inventors: Andy Scherer, MAPLE GROVE, MN (US); Sooneon Bae, MAPLE GROVE, MN (US); Ryan Sotak, COLORADO SPRINGS, CO (US)

(21) Appl. No.: 18/871,175

(22) PCT Filed: May 26, 2023
(86) PCT No.: PCT/EP2023/064147

§ 371 Date: Dec. 3, 2024

Related U.S. Application Data
(60) Provisional application No. 63/350,443, filed on Jun. 9, 2022.

(51) Int. Cl. A61B 17/3207; A61B 17/00

(52) U.S. Cl. CPC A61B 17/320758; A61B 2017/00867

(57) ABSTRACT

An intravascular therapy device (10) includes an intravascular catheter (12); a helical coil (14) disposed at a distal end (13) of the intravascular catheter; at least one cutter (16) mounted on the helical coil; and a rotary control (18) disposed at a proximal end (15) of the intravascular catheter and operatively connected to rotate the helical coil.

### FIELD

The following relates generally to the catheter arts, vascular therapy, lesion treatment arts, and related arts.

### BACKGROUND

Venous thromboembolism, which includes deep venous thrombosis (DVT), is a major contributor to the global disease burden and is the third most common cardiovascular pathology after coronary artery disease and stroke. Lower extremity DVT (LEDVT) can block the venous lumen and leads to venous congestion, swelling, and lower extremity venous valve function damage, resulting in post-thrombotic syndrome (PTS).

Standard treatment of venous obstruction includes the use of balloons, stents, lytics, aspiration and mechanical thrombectomy. Balloons and stents are inexpensive and time efficient treatment options but do not remove the obstruction from the vessel, which can lead to reoccurrence of the disease. Also, stents are typically not considered as a treatment option below the lesser trochanter due to poor long term patency in this anatomy. Lytics, aspiration, and mechanical thrombectomy treatments effectiveness drop significantly with the age of clot becoming ineffective for chronic obstructions.

The following discloses certain improvements to overcome these problems and others.

### SUMMARY

In some embodiments disclosed herein, an intravascular therapy device includes an intravascular catheter; a helical coil disposed at a distal end of the intravascular catheter; at least one cutter mounted on the helical coil; and a rotary control disposed at a proximal end of the intravascular catheter and operatively connected to rotate the helical coil.

In some embodiments disclosed herein, a vascular therapy method includes inserting an intravascular catheter into a blood vessel to position a helical coil at a distal end of the intravascular catheter proximate to a clot; and rotating the helical coil using a rotary control at a proximal end of the intravascular catheter to cause the helical coil to screw into the clot and to cause the clot to be cut by at least one cutter mounted on the helical coil.

In some embodiments disclosed herein, an intravascular therapy device includes a helical coil having a stiffness effective for rotation into engagement with a vascular occlusion and a flexibility effective to provide bending of the helical main body to conform with a path of a blood vessel in which the vascular occlusion is disposed; and at least one cutter mounted on the helical main body and configured to cut into the vascular occlusion.

One advantage resides in providing a debulking tool for a clot.

Another advantage resides in providing a helical debulking tool for debulking a clot in lieu of an angioplasty balloons or a stent.

Another advantage resides in providing a helical debulking tool having one or more cutters for debulking a clot.

Another advantage resides in providing a helical debulking tool that can treat multiple locations of a clot not accessible by a stent.

Another advantage resides in providing a helical debulking tool that includes at least one tangential cutter for cutting a plug of clot material and at least one diametrical cutter to break up the plug contained within the helix.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a vascular therapy device in accordance with the present disclosure.

FIGS. 2-4 diagrammatically illustrate embodiments of a helical coil of the device of FIG. 1.

FIG. 5 diagrammatically illustrates a method of performing a vascular therapy method using the device of FIG. 1.

### DETAILED DESCRIPTION

The following relates to a debulking tool for use in intravascular therapy. The tool has a helical main body which has sufficient stiffness to be rotated into engagement with a vascular clot and sufficient flexibility to provide some bending to conform with the blood vessel path. At least one cutter is mounted on the helix. The helix advantageously enables controlled and driven entry into the clot by rotating the shaft on which the helix is mounted, and the at least one cutter cuts through the clot material as the tool is rotated. Advantageously, the design removes the clot as a solid core that is retained within the helix so that it is removed with the tool after the debulking procedure is complete.

Some helical debulking tool embodiments disclosed herein include two cutters: a tangential cutter disposed across two neighboring helical turns of the helix, and a diametrical cutter disposed across the diameter of a single helical turn. The diagrammatical cutter can be optional—if provided it serves to break up the plug contained within the helix.

The helix of the debulking tool can be made of various materials, e.g. stainless steel, nitinol, or a shape memory polymer for example. The latter two designs have the advantage that the helix can be set in the expanded helical shape but can be stored in the catheter as a single wire formed by unrolling the helix or as a compacted helix, and the originally set helix shape is then recovered upon deployment inside the blood vessel.

In an embodiment suitable for manual operation, the surgeon has a knob located at the proximal end of the catheter, outside of the patient, that can be rotated to screw the helix into the clot. The knob may include gearing to provide force multiplication, e.g. N turns of the physical knob could produce MN turns of the helix where M is greater than 1 for force multiplication (and less than 1 if the force is greater at the helix).

The debulking tool can be used for venous clots, and also for arterial clots. The debulking tool is expected to provide faster debulking than laser ablation.

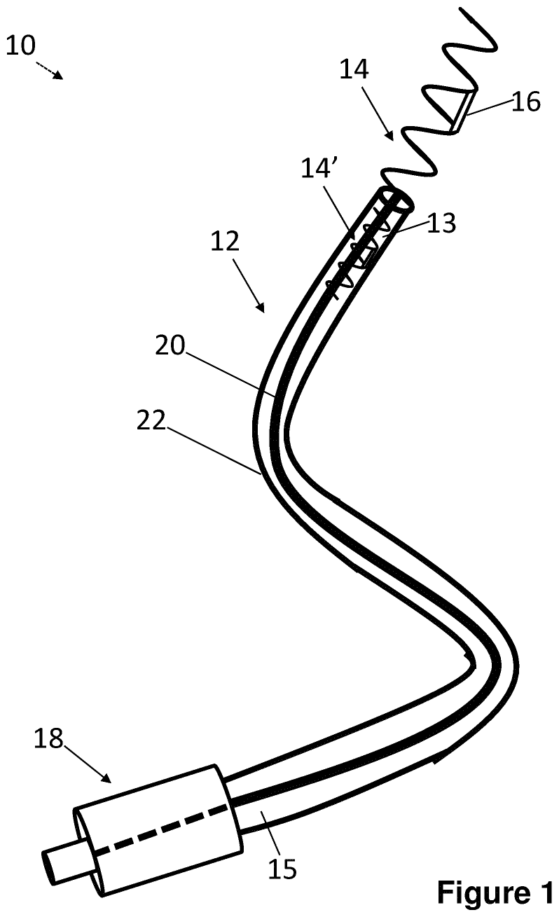

With reference to FIG. 1, an illustrative vascular therapy device 10 is diagrammatically shown. As shown in FIG. 1, the vascular therapy device 10 is insertable into a blood vessel for treating a lesion (or a clot, or an occlusion, and so forth) in the blood vessel. The vascular therapy device 10 includes, for example, an intravascular catheter 12, a helical coil 14 disposed at a distal end 13 of the intravascular catheter 12, at least one cutter 16 mounted on the helical coil 14, and a rotary control 18 disposed at a proximal end 15 of the intravascular catheter 12. It is noted that FIG. 1 is not drawn to scale, and that the intravascular catheter 12 can have a length suitable to insert the catheter 12 into a blood vessel to deliver the distal end 13 through the vasculature along a (possibly tortuous) path to a treatment site, with the length of the catheter 12 being sufficient so that the rotary control 18 still remaining outside of the patient when the distal end 13 reaches the treatment site. Although not shown, it is contemplated to include a safety cage disposed around the deployed cutting tool to ensure the cutter(s) 16 do not cut into the inner wall of the blood vessel. In some examples, the helical coil 14 can include a coating. In other examples, a lytic solution can be flowed through the intravascular catheter 12 and the helical coil 14 when the intravascular catheter 12 includes a hypotube and a sealed tip.

The rotary control 18 is operatively connected to rotate the helical coil 14. The rotary control 18 can be manually rotated by a user, or can be motorized with a motor (not shown). As shown in FIG. 1, the illustrative intravascular catheter 12 includes a control wire or cable 20 (referred to from here on as a control wire 20 for brevity) disposed within a sheath 22 that coaxially surrounds the control wire 20. The rotary control 18 is operatively connected to rotate the helical coil 14 by the control wire 20. To do so, the control wire 20 is longitudinally movable within the sheath 22 to selectably (i) withdraw the helical coil 14 into the sheath 22 (shown in FIG. 1 as withdrawn helical coil 14′ located within the distal end of the sheath 22) and (ii) deploy the helical coil 14 out of the sheath 22 (shown in FIG. 1 as the deployed helical coil 14 located outside of the sheath 22). This is merely an illustrative example, and more generally the catheter 12 can have other features not shown in FIG. 1, such as a guidewire lumen running the entire length of the catheter 12 for over the wire (OTW) delivery along a guidewire that is pre-inserted into the blood vessel along the path to the treatment site, or a shorter guidewire lumen with an exit port in a rapid exchange (RX) catheter design. By way of further illustration, other contemplated variants include addition of an ultrasound transducer at or near the distal end 13 for imaging the treatment site, addition of one or more radiopaque markers on the catheter 12 to enable visualization by a suitable interventional imaging modality, and/or so forth. For example, an acoustic wave can be transmitted through an ultrasound transducer to further treat a lesion. In some embodiments, the proximal end may be connected with a vacuum pump (not shown) to implement vacuum aspiration via the sheath 22 to remove clot material cut away by the cutter 16.

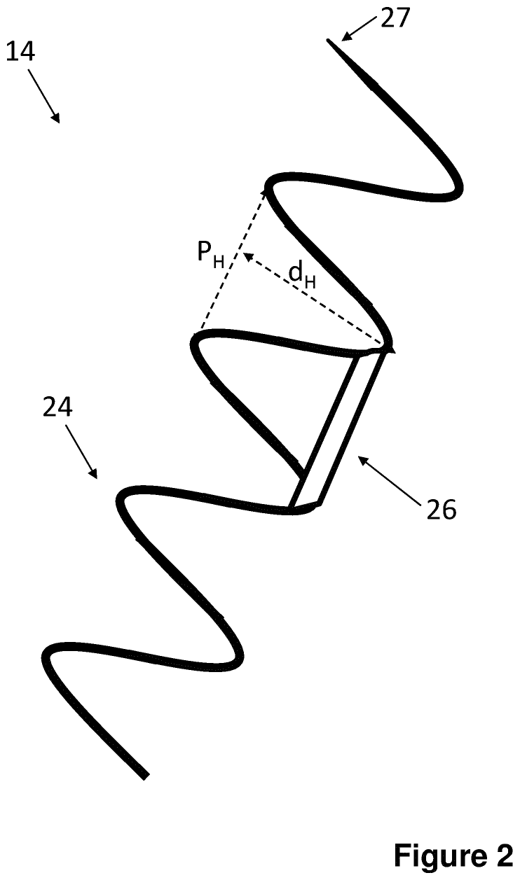

With continuing reference to FIG. 1, FIGS. 2-4 show example embodiments of the helical coil 14 and the cutter 16. The helical coil 14 (and the at least one cutter 16) can comprise, for example, stainless steel or Nitinol. The helical coil 14 comprises a plurality of turns 24 that form the helical coil 14 as a helix. The helical coil 14 has stiffness effective for the helical coil 14 to be screwed into a clot by rotation of the helical coil 14 by the rotary control 18. In other words, the helical coil 14 is sufficiently stiff to maintain its helical shape under the forces acting on the coil 14 as it is threaded into clot material. For example, in one embodiment the helical coil 14 can comprise a wire of surgical stainless steel shaped into a helix, in which the stainless steel wire has a wire diameter sufficiently large to provide the desired stiffness. The requisite stiffness is also dependent on the nature of the clot being treated, e.g. a harder calcified clot may require the helical coil 14 be made of stiffer wire than a clot of a softer or more pliant material.

In the embodiment shown in FIG. 2, the cutter 16 comprises one (as illustrated) or more tangential cutters 26 connecting across adjacent turns 24 of the helical coil 14. This cutter 26 is tangential as it is along a tangent of the helix. The tangential cutter is thus positioned at (or at about) the radius of the helix. If the diameter of the helix is d_{H} (corresponding radius r_{H}=0.5×d_{H}) and the blood vessel diameter is d_{BV} (corresponding radius r_{BV}=0.5×d_{BV}), then the tangential cutter 26 is positioned at a position r_{BV}-r_{H} away from the inner blood vessel wall. Hence, if d_{H} is just slightly smaller than d_{BV} then the tangential cutter 26 is positioned to cut out a core of diameter d_{H} which will remove most of the clot material, except for the outermost d_{BV}-d_{H} annulus of the clot. Put another way, the tangential cutter 26 is arranged to cut a core of clot material of core diameter about equal to d_{H}. Advantageously, rotation of the helical coil 14 using the rotary control 18 (or, in another embodiment, a motor) provides screwing effect that drives the helical coil 14 into the clot and this in turn drives the tangential cutter 26 in a cutting motion to cut away the core of clot material. To facilitate initial engagement of the helical coil 14 into the clot material, in the illustrative embodiments the helical coil 14 has a tapered tip 27 that forms a leading point that more easily engaged into the clot material as the first turn of the helical coil 14 encounters the clot. In another example, the leading point can also be pointed slightly inward to the center of the helical coil 14, or also the helical coil 14 can be formed at a slightly smaller diameter at the end to help ensure that the leading point will not perforate the vessel wall.

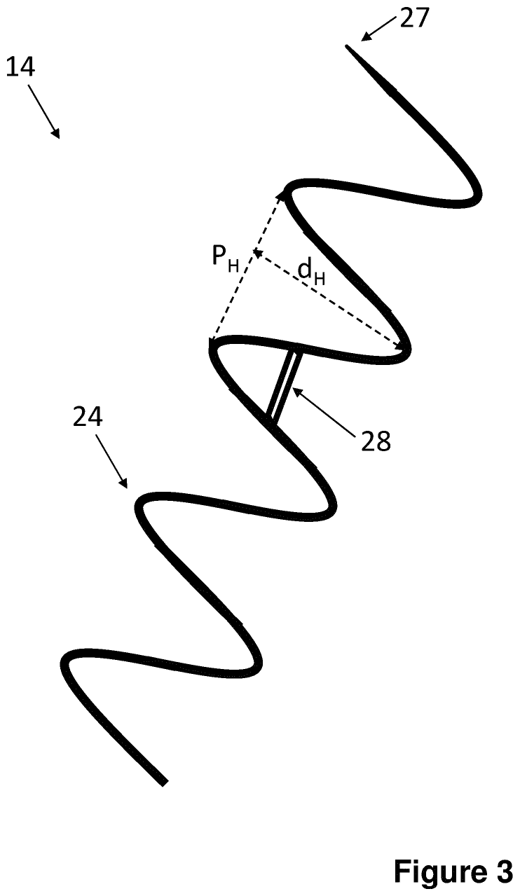

In the embodiment shown in FIG. 3, the helical coil 14 including tapered tip 27 is again included, but in this embodiment the cutter 16 comprises one (as illustrated) or more spanning cutters 28 connecting across a single turn 24 of the helical coil 14. In some embodiments, the spanning cutter 28 is positioned to intersect the central axis of the helix of the helical coil 14 in which case the spanning cutter 28 spans a diameter of the helix and may be referred to as a diametrical cutter 28. However, in other embodiments the spanning cutter 28 may pass near, but not directly intersect, the central axis of the helix. Unlike the tangential cutter 26 of the embodiment of FIG. 2, the spanning cutter 28 of the embodiment of FIG. 3 is not positioned at the outer diameter du of the helix, and as such may be less effective at cutting clot material close to the blood vessel wall. However, the spanning cutter 28 can be effective at debulking clot material more centrally located within the blood vessel, for example in the case of a clot forming a complete or near complete blockage of blood flow through the blood vessel.

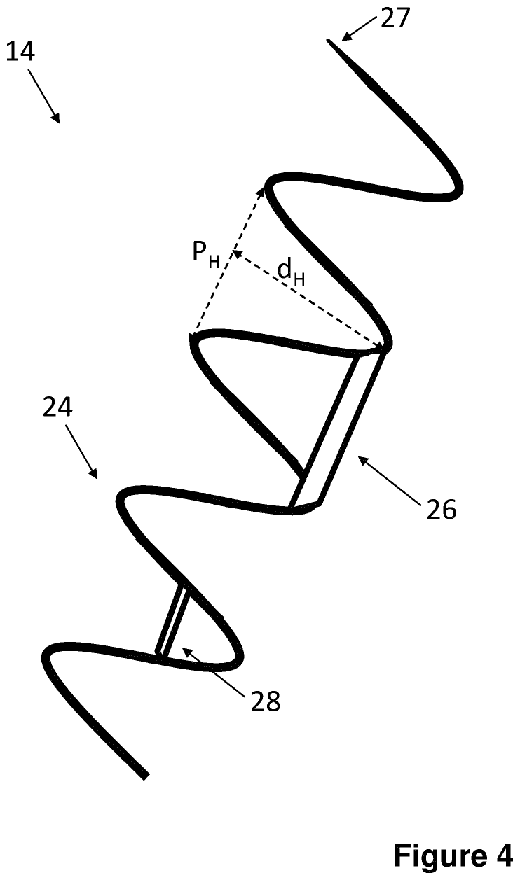

The embodiment shown in FIG. 4 combines the embodiments of FIGS. 2 and 3, so that the cutter 16 comprises both a tangential cutter(s) 26 and a spanning cutter(s) 28. In the illustrative example, the tangential cutter(s) 26 is located closed to the tapered tip 27 of the helical coil 14 that first engages the clot than the spanning cutter(s) 28. In this way, the tangential cutter(s) 26 operate first to cut away a core of clot material, and then the “downstream” spanning cutter(s) 28 can debulk or cut a spiral through the clot core that was disengaged from the blood vessel wall by the tangential cutter(s) 26. This latter debulking or spiral-cutting of the clot core makes it more easily removed by a process such as vacuum aspiration. Thus, in the embodiment of FIG. 4, the plurality of turns 24 allows the helical coil 14 to be threaded through the clot like a corkscrew. Rotating the helical coil 14 drives it forward at a consistent advancement rate based on the pitch of the helical coil 14. The tangential cutter(s) 26 cuts around the perimeter that is traced out by the helical frame. The tracing of the tangential cutter(s) 26 around the perimeter creates a plug of clot material that can be removed from the bulk clot material. The spanning cutter(s) 28 diametrically scores the plug into a spiral shape so that it may be easier to capture and remove from the tool and the body as needed.

The embodiments of FIG. 2-4 are merely illustrative, and numerous variants are contemplated. The cutters can be otherwise placed on the helical coil 14. Also, the cutter(s) 16 can comprise blades, blunt dissection surfaces, serrated edges, various combinations thereof, and/or so forth. Furthermore, the helical pitch P_{H} of the helical coil 14 can be variously designed. For example, a small helical pitch P_{H} can provide more mechanical advantage and increase the cutting force, at the cost of a slower clot removal process as more rotations of the small-pitch helical coil will be required in order for the cutter to travel a given distance along the blood vessel. By contrast, a large helical pitch P_{H} can provide faster cutting, but with less mechanical advantage.

The vascular therapy device 10 is assembled in one embodiment by attaching the cutter(s) 16 to the helical coil 14, for example by welding or the like. The tangential cutter(s) 26 is attached tangentially to the helical coil 14 and the spanning cutter(s) 28 is attached in a diametrical orientation. The cutter(s) 16 are in some embodiments attached a couple turns or so of the helical coil 14 away from the leading tip 27 of the helical coil 14. This provides the helical coil 14 a couple of turns 24 to establish its path into the clot, stabilize the coring element direction, and during continued rotation the initial couple of turns of the helix now embedded into the clot material helps to pull the rest of the vascular therapy device 10 forward before the cutter(s) 16 start cutting into the clot.

With reference back to FIG. 1, the vascular therapy device 10 is mounted at the distal end 13 of the intravascular catheter 12 to provide intravascular access to the desired portion of the anatomy. The intravascular therapy device 10 is advanced by the operator rotating the rotary control 18 that is attached to the helical coil 14. As the vascular therapy device 10 is rotated it advances forward at a speed related to the pitch P_{H} of the helical coil 14. The cutter(s) 16 (and especially the tangential cutter(s) 26) of the helical coil 14 cuts a plug free from the bulk clot as it advances. Once the helical coil 14 has advanced through the clot and the cutter(s) 16 has separated the plug from the bulk clot, the plug will remain inside of the helical coil 14. When the vascular therapy device 10 is removed from the blood vessel, the plug will come with it. Once outside of the body, the plug can be removed from the vascular therapy device 10 and the vascular therapy device 10 can be re-entered into the clot to remove additional material if desired.

The spanning cutter(s) 28 is effective to alter the form of the plug that is created. If one solid plug is acceptable from a performance standpoint, only a single tangential cutters 26 can be used, as in the embodiment of FIG. 2. If slicing the plug into a spiral shape becomes beneficial for removing the plug from the helical coil 14, then a combination of tangential cutter(s) 26 and spanning cutter(s) 28 is beneficial to achieve this effect.

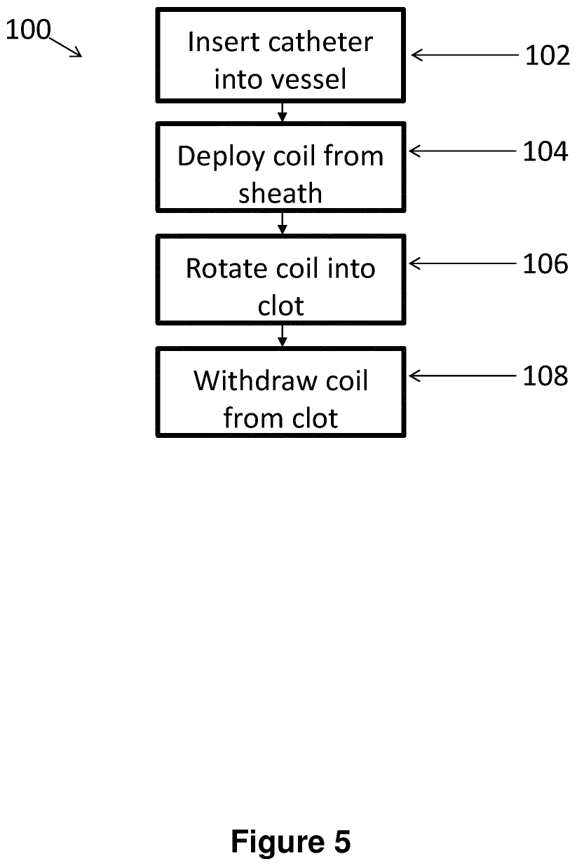

Referring to FIG. 5, an illustrative embodiment of an intravascular therapy method 100 using the intravascular therapy device 10 is diagrammatically shown as a flowchart. At an operation 102, the intravascular catheter 12 is inserted into a blood vessel to position the helical coil 14 proximate to a clot in the blood vessel. The helical coil 14 is withdrawn into the sheath 22 during the inserting (corresponding to withdrawn helical coil 14′ of FIG. 1). At an operation 104, the helical coil 14 is deployed from the sheath 22 by longitudinal movement of the control wire 20 via rotation of the rotary control 18. The deployed helical coil is shown in FIG. 1 as helical coil 14. At an operation 106, the deployed helical coil 14 is rotated using the rotary control 18 to cause the helical coil 14 to screw into the clot, and to cause the clot to be cut by the cutter(s) 16. In one example, the rotating causes a clot core to be cut out of the clot by the tangential cutter(s) 26. In another example, the rotating causes a clot core to be cut out of the clot by the spanning cutter(s) 28. At an operation 108, the helical coil 14 is withdrawn back into the sheath 22 by longitudinal movement of the control wire 20 via rotation of the rotary control 18. In some examples, the intravascular therapy device 10 can comprise a vessel lumen protection cage, while another additional debulking device is in use at the same time after helical coil while other additional debulking device is in use at the same time after helical coil has in place (which can be performed between the operations 106 and 108).

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An intravascular therapy device, comprising:
an intravascular catheter;
a helical coil disposed at a distal end of the intravascular catheter;
at least one cutter mounted on the helical coil; and
a rotary control disposed at a proximal end of the intravascular catheter and operatively connected to rotate the helical coil.

2. The intravascular therapy device of claim 1, wherein the at least one cutter includes at least one tangential cutter connecting across adjacent turns of the helical coil.

3. The intravascular therapy device of claim 1, wherein the at least one cutter includes at least one spanning cutter connecting across a single turn of the helical coil.

4. The intravascular therapy device of claim 1, wherein the at least one cutter includes:
at least one tangential cutter connecting across adjacent turns of the helical coil; and
at least one spanning cutter connecting across a single turn of the helical coil;
wherein the at least one tangential cutter is located closer to a tip of the helical coil than the at least one spanning cutter.

5. The intravascular therapy device of claim 1, wherein the helical coil has stiffness effective for the helical coil to be screwed into a clot by rotation of the helical coil by the rotary control of the intravascular therapy device.

6. The intravascular therapy device of claim 1, wherein the helical coil comprises stainless steel or Nitinol.

7. The intravascular therapy device of claim 1, wherein the intravascular catheter includes:
a control wire or cable; and
a sheath coaxially surrounding the control wire or cable;
wherein the rotary control is operatively connected to rotate the helical coil by the control wire or cable.

8. The intravascular therapy device of claim 7, wherein the control wire or cable is longitudinally movable within the sheath to selectably (i) withdraw the helical coil into the sheath and (ii) deploy the helical coil out of the sheath.

9. A vascular therapy method, comprising:
inserting an intravascular catheter into a blood vessel to position a helical coil at a distal end of the intravascular catheter proximate to a clot; and
rotating the helical coil using a rotary control at a proximal end of the intravascular catheter to cause the helical coil to screw into the clot and to cause the clot to be cut by at least one cutter mounted on the helical coil.

10. The vascular therapy method of claim 9, wherein the rotating causes a clot core to be cut out of the clot by a tangential cutter connecting across adjacent turns of the helical coil.

11. The vascular therapy method of claim 10, wherein the rotating further causes the clot core to be debulked by a spanning cutter connecting across a single turn of the helical coil.

12. The vascular therapy method of claim 9, wherein the intravascular catheter includes a control wire or cable surrounded by a sheath, the rotary control is operatively connected via the control wire or cable to rotate the helical coil, the helical coil is withdrawn into the sheath during the inserting, and the method further comprises:
after the inserting and before the rotating, deploying the helical coil from the sheath by longitudinal movement of the control wire or cable within the sheath; and
after the rotating, withdrawing the helical coil back into the sheath by longitudinal movement of the control wire or cable within the sheath.

13. An intravascular therapy device, comprising:
a helical coil having a stiffness effective for rotation into engagement with a vascular occlusion and a flexibility effective to provide bending of the helical main body to conform with a path of a blood vessel in which the vascular occlusion is disposed; and
at least one cutter mounted on the helical main body and configured to cut into the vascular occlusion.

14. The intravascular therapy device of claim 13, wherein the helical coil comprises a plurality of turns;
wherein the at least one cutter is disposed on one or more of the turns.

15. The intravascular therapy device of claim 13, wherein the at least one cutter comprises:
a cutter disposed across a diameter of a single turn of the helical coil.

16. The intravascular therapy device of claim 15, wherein the at least cutter further comprises:
a second cutter disposed across neighboring turns of the helical coil.

17. The intravascular therapy device of claim 13, further comprising:
an intravascular catheter;
a rotary control operatively connected to rotate the helical coil;
wherein the helical coil is disposed at a distal end of the intravascular catheter and the rotary control is disposed at a proximal end of the intravascular catheter.

18. The intravascular therapy device of claim 17, wherein the intravascular catheter includes:
a control wire or cable; and
a sheath coaxially surrounding the control wire or cable;
wherein the rotary control is operatively connected to rotate the helical coil by the control wire or cable.

19. The intravascular therapy device of claim 18, wherein the control wire or cable is longitudinally movable within the sheath to selectably (i) withdraw the helical coil into the sheath and (ii) deploy the helical coil out of the sheath.

20. The intravascular therapy device of claim 13, wherein the helical coil comprises Nitinol.
